(19) United States
(12) Patent Application Publication
(10) Pub. No.: US 2009/0216158 A1
(43) Pub. Date: Aug. 27, 2009

(54) ULTRASOUND AND TISSUE REPAIR

(76) Inventors: ANDREW JAMES HARRISON, Huntington (GB); CLAIRE MAHONEY, (US); MARTIN HUMPHRIES, Brernhall (GB); MARK BASS, West Didsbury (GB)

Correspondence Address: DIANA HOUSTON;SMITH & NEPHEW, INC., 1450 BROOKS ROAD, MEMPHIS, TN, 38116, US

(21) Appl. No.: 12/390,065

(22) Filed: Feb. 20, 2009

(30) Foreign Application Priority Data
Feb. 22, 2008 (GB) 0803272.4

(51) Int. Cl. A61N 7/00

(52) U.S. Cl. 601/2

(57) ABSTRACT

Methods of using an ultrasound signal to promote tissue repair by causing cells to form focal adhesions without the involvement of syndecans, in particular syndecan-1, syndecan-4 or PKCα are disclosed.

### CROSS-REFERENCE TO RELATED APPLICATIONS

This application claims priority from UK provisional application No. 0803272.4 filed on 22 Feb. 2008 and entitled “Ultrasound and Tissue Repair”, the entire contents of which are hereby incorporated by reference.

### BACKGROUND OF THE INVENTION

1. Field of the Invention

The invention relates to methods of using an ultrasound signal to promote tissue repair by promoting cells to form focal adhesions without the involvement of syndecans or PKCα.

2. Related Art

Therapeutic approaches to the treatment of tissue wounds and bone fractures differ from the treatment of pathogen infections, tumor development or genetic disorders. Effective wound therapy should be non-invasive, by definition, to avoid causing further tissue damage and should augment the bodies existing healing processes without inducing excessive cell proliferation or extracellular matrix (ECM) synthesis that lead to scarring. The advantages of accelerating wound closure are significant as they not only improve patient comfort, but also reduce the risk of infection following injury or surgery. A recently developed approach to this problem has been the application of low-intensity, pulsed ultrasound to a wound area, through transducers coupled to the skin with a water-based gel. The physiological benefits of this approach to bone fracture healing have been startling, with the healing times of tibial and radial fractures reduced by 38% (Heckman et al., 1994; Kristiansen et al., 1997). Ultrasound is particularly beneficial to the treatment of non-union fractures, which do not heal without intervention and are traditionally treated by pinning the bone, surgically. Ultrasound treatment results in the closure of 85% of non-union fractures, a figure that is similar to the surgical success rate (68-96% of cases), but avoids the complications associated with surgery (Gebauer et al., 2005).

The processes of tissue healing: proliferation, migration, differentiation and ECM synthesis each depend on signals activated during cell adhesion to the ECM. Cell adhesion is mediated by engagement of transmembrane ECM-receptors of the integrin and syndecan families. Integrins interact with ECM molecules, such as fibronectin, through a direct protein-protein association (Arnaout et al., 2005), whereas syndecans bind to the polybasic regions of ECM molecules via the glycosaminoglycan chains, covalently attached to the syndecan extracelullar domain (Bernfield et al., 1999).

There are examples of functional synergy between a number of integrin-syndecan pairs (Morgan et al., 2007), but the best characterized is the co-operation between α₅β₁ integrin and syndecan-4 during adhesion to fibronectin. Notably, both α₅β¹ integrin and syndecan-4 are over-expressed on fibroblasts and endothelial cells surrounding a dermal wound (Cavani et al., 1993; Gallo et al., 1996). There is extensive evidence that co-operative signaling by this pair of receptors is necessary for the formation of vinculin-containing adhesion complexes during spreading on fibronectin (Bloom et al., 1999; Woods et al., 1986).

Syndecans play a key role in wound healing. For example, the disruption of syndecan-1 and -4 has been demonstrated to impair wound healing in mice (Echtermeyer et al 2001; Stepp et al 2002) and syndecans are up-regulated in wounds (Gallo et al, 2006; Elenius et al, 1991).

The mechanism of co-operation between the two receptors has been characterized and the small GTPase Rac1, which regulates membrane protrusion (Ridley, 2006), has been identified as a convergence point (Bass et al., 2007b; Del Pozo et al., 2004). Syndecan-4 determines GTP-loading of Rac1 via PKC□ (Bass et al., 2007b), while α₅β₁ integrin regulates the association of GTP-bound Rac1 with the plasma membrane, which is necessary for the activation of downstream effectors (Del Pozo et al., 2004).

### SUMMARY OF THE INVENTION

We have found that ultrasound has an effect on adhesion-dependent signaling and have demonstrated that ultrasound can drive focal adhesion formation on a minimal integrin-binding ECM. Ultrasound acts by activating Rac1 and is capable of doing so in the absence of activation or expression of syndecan-4 or PKCα. As such we find that ultrasound stimulation dispenses with the necessity of the syndecan-4 signaling axis for Rac1 regulation. This is of particular therapeutic advantage in individuals with a compromised syndecan-4 or PKCα expression.

We have identified that ultrasound, particularly a low-intensity pulsed ultrasound signal, can trigger the activation of the GTPase Rac1 resulting in the formation of focal adhesions through a mechanism that is independent of the syndecan-4/PKCα signaling cascade. This is particularly advantageous in subjects in which the syndecan-4/PKCα signaling cascade is compromised, for example where there are deficiencies in syndecan-4 and/or PKCα

According to a first aspect of the invention there is provided a method of activating a member of the Rho kinase family in a subject by exposing the subject to an ultrasound signal.

According to a second aspect of the invention there is provided a method of promoting tissue repair in a subject by activating the GTPase Rac1, the method comprising the step of exposing the tissue to an ultrasound signal.

According to a third aspect of the invention there is provided a method of promoting the formation of focal adhesions in a subject by exposing the subject to an ultrasound signal.

According to a fourth aspect of the invention there is provided a method of determining the likelihood of a deficient wound healing response in an individual, the method comprising the steps of: (i) determining the concentration of a syndecan in a sample of bodily fluid taken from an individual; and (ii) comparing the concentration with a pre-determined cut-off value, said cut-off value being chosen to exclude concentrations of a syndecan associated with an optimal wound healing response in an individual, wherein a concentration below the cut-off value is indicative of the likelihood of a sub-optimal wound healing response.

According to a fifth aspect of the invention there is provided a method of enhancing the wound healing response in an individual, the method comprising the steps of: (i) determining the concentration of a syndecan in a sample of bodily fluid taken from an individual; (ii) comparing the concentration with a pre-determined cut-off value, said cut-off value being chosen to exclude concentrations of a syndecan associated with an optimal wound healing response in an individual, wherein a concentration below the cut-off value is indicative of the likelihood of a sub-optimal wound healing response, and (iii) exposing the wound to ultrasound if the concentration of a syndecan in the bodily fluid is below the cut-off value.

According to a sixth aspect of the invention there is provided a method of enhancing the wound healing response in an individual, the method comprising the steps of: (i) determining the concentration of a PKCα in a sample of bodily fluid taken from an individual; (ii) comparing the concentration with a pre-determined cut-off value, said cut-off value being chosen to exclude concentrations of a PKCα associated with an optimal wound healing response in an individual, wherein a concentration below the cut-off value is indicative of the likelihood of a sub-optimal wound healing response, and (iii) exposing the wound to ultrasound if the concentration of a syndecan in the bodily fluid is below the cut-off value.

According to a seventh aspect of the invention there is provided a method of enhancing the wound healing response in an individual, the method comprising the steps of: (i) determining the concentration of a PKCα in a sample of bodily fluid taken from an individual; (ii) comparing the concentration with a pre-determined cut-off value, said cut-off value being chosen to exclude concentrations of a PKCα associated with an optimal wound healing response in an individual, wherein a concentration below the cut-off value is indicative of the likelihood of a sub-optimal wound healing response, and (iii) exposing the wound to ultrasound if the concentration of a PKCα in the bodily fluid is below the cut-off value.

In embodiments of the first aspect of the invention the Rho kinase is GTPase Rac1. It has been demonstrated that the ultrasound signal can directly activate GTPase Rac1.

Alternatively the ultrasound signal can activate a component of the syndecan 4/PKCα signaling pathway which is upstream of the GTPase Rac1. In embodiments of the invention, this upstream component is not syndecan 4 or PKCα.

The promotion of tissue healing by ultrasound according to the second aspect of the invention is via the activation of the GTPase Rac1, which is independent of the syndecan 4/PKCα signaling pathway. This activation of Rac1 results in the formation of focal adhesions, a key step in the tissue repair process.

In embodiments of the fourth or fifth aspects of the invention, the syndecan is, for example, syndecan-1 or syndecan-4.

Syndecan-1 and -4 and PKCα are ubiquitously expressed within the body and as such their concentration can be determined in any bodily fluid, for example; blood, serum or plasma. The ectoderms of syndecan-1 and -4 are shed around wounds and as such the measurement of levels of syndecan-1 and -4 ectoderms in the tissue fluid surrounding a wound are directly indicative of the wound healing process. Measurement of the ectoderms can be achieved by a two-stage punch biopsy.

An individual is considered likely to exhibit an impairment of the wound healing response if the expression of syndecan-1 or -4 or PKCα is less than 50% of that of an individual with an optimal wound healing response.

An individual is considered likely to exhibit a severe impairment of the wound healing response if the expression of syndecan-1 or -4 or PKCα is less than 25% of that of an individual with an optimal wound healing response.

In embodiments of the invention the ultrasound intensity is at least 25 mWcm-2, for example between about 30 mWcm-2 and 50 mWcm-2.

The ultrasound signal can be pulsed. A suitable signal is characterized by a 1.5 MHz wave frequency and a 1 KHz pulse frequency.

The treatment regime can vary depending on the individuals needs and is at the discretion of the medical personnel. For example, the treatment regime can consist of repeated exposures to ultrasound within a 24 hour period. Alternatively, the treatment regime can consist of a daily single exposure to ultrasound.

The ultrasound signal is advantageously administered to the wound for at least about eight minutes in every treatment.

The ultrasound signal is advantageously administered to the wound for at least between about eight minutes and about twenty minutes in every treatment

The ultrasound signal is advantageously administered to the wound for at least about 8 minutes, or at least about 9 minutes, or at least about 10 minutes, or at least about 11 minutes, or at least about 12 minutes, or at least about 13 minutes, or at least about 14 minutes, or at least about 15 minutes, or at least about 16 minutes, or at least about 17 minutes, or at least about 18 minutes, or at least about 19 minutes, or at least about 20 minutes.

The subject can be a human or a non-human animal.

It is envisaged that the methods of the present invention can be used to repair any tissue in the body to which it is possible to administer an ultrasound signal.

In embodiments of the methods of the invention the tissue is cartilage. For instance, the methods can be used to repair focal cartilage defects

In embodiments of the methods of the invention the tissue is bone. For instance, the methods can be used to repair bone fractures.

In embodiments of the methods of the invention the tissue is tendon. For instance, the methods can be used to treat chronic tendonopathy.

In embodiments of the methods of the invention the tissue is ligament. For instance, the methods can be used to repair tears of ligaments such as the anterior cruciate ligament.

In embodiments of the methods of the invention the tissue is muscle. For instance, the methods can be used to repair muscle damage.

In embodiments of the methods of the invention the tissue is a nerve tissue. For instance, the methods can be used to treat neuropathy.

In embodiments of the methods of the invention the tissue is a spinal disc. For instance, the methods can be used to repair spinal vertebral disc regeneration.

In embodiments of the methods of the invention the tissue is skin. For instance, the methods can be used wounds. For example, surgical wounds, burns, venous ulcers.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The accompanying drawings, which are incorporated in and form a part of the specification, illustrate the embodiments of the present invention and together with the written description serve to explain the principles, characteristics, and features of the invention. In the drawings:

FIG. 1: Ultrasound stimulates focal adhesion formation independently of syndecan-4. (A) Schematic representation of the ultrasound wave form. Wild-type (B−C) and syndecan-4-null MEFs (D−E) were spread on 50K for 2 hours before stimulating with syndecan-4 ligand (H/0) for 40 minutes or ultrasound for 20 minutes. Fixed cells were stained for vinculin (green) and actin (red), bar=5 μm. Total focal adhesion areas of 20-25 cells were measured using image J software (C+E). (F) Focal adhesion area of wild-type MEFs stimulated with a range of ultrasound intensities. (G) Focal adhesion area of wild-type MEFs stimulated with ultrasound for a range of durations. Error bars indicate SEM, asterisks indicate significance values; *=p<0.05, **=p<0.01 and ***=p<0.001 when compared to un-stimulated cells. Images and analyses are representative of experiments performed on three separate occasions.

FIG. 2: Ultrasound does not elicit its effect through integrin activation. (A) Flow cytometry of K562 cells following ultrasound stimulation using monoclonal antibody 12G10 which recognises an activation epitope of human β₁ integrin. 1 mM manganese was used as a positive control to drive integrin activation. (B) Rate of integrin-mediated spreading of control (crosses) or ultrasound-stimulated (circles) MEFs. The area of 150 cells was measured using Image J software, error bars indicate SEM. (C+D) The areas of MEFs plated onto poly-L-lysine, H/0 or 50K for 120 minutes were unaffected by stimulation with ultrasound, bar=50 μm. (E) Fibroblasts spread on 50K or the inhibitory β₁ monoclonal antibody, mab13, and stimulated with ultrasound were stained for vinculin (green) and actin (red), bar=5 μm. Each result is representative of three independent experiments.

FIG. 3: Ultrasound causes syndecan-4-independent Rac1 regulation. Rac1 activity was measured by effector pull-down assays in combination with quantitative Western blotting using fluorophore-conjugated antibodies. Wild-type MEFs (A−B) and syndecan-4-null MEFs (C) were pre-spread on 50K for 2 hours and stimulated with H/0 (A) or ultrasound (B−C) over a 60 minute time-course, before preparing lysates. Equivalent loading between experiments was confirmed by blotting crude lysates for total vinculin. Graphs are representative of 5-10 individual experiments, error bars indicate SEM and asterisks indicate significance values (p<0.05) when compared to un-stimulated cells.

FIG. 4: Ultrasound-stimulated Rac1 regulation occurs independently PKCα. Rac1 activity was measured by effector pull-down assays in combination with quantitative Western blotting using fluorophore-conjugated antibodies. Wild-type MEFs transfected with a non-targeting control siRNA (A), an siRNA specific to PKCα (B) or treated with 200 nM BIM for 30 min before and throughout stimulation (C) were prespread on 50K for 2 hours and stimulated with ultrasound over a 60 minute time-course, before preparing lysates. Equivalent loading between experiments was confirmed by blotting crude lysates for total vinculin. Error bars indicate SEM and asterisks indicate significance values (p<0.05) when compared to un-stimulated cells. (D) Expression levels of PKCα, PKCδ, or PKCε following RNAi. Analyses are representative of 5-10 independent experiments.

FIG. 5: Ultrasound-induced focal adhesion formation is independent of the syndecan-4-PKCα signaling axis. MEFs transfected with a non-targeting control siRNA (A), an siRNA specific to PKCα (C) or treated with 200 nM BIM throughout (E) were pre-spread on 50K for 2 hours prior to stimulation with H/0 or ultrasound, followed by fixing and staining cells with vinculin (green) and actin (red). Bar=5 μm. Focal adhesion area was quantified for 20 cells per condition using Image J software (B, D, F and G). Error bars indicate SEM, RNAi and BIM results are representative of two and three independent experiments respectively.

### DETAILED DESCRIPTION OF THE EMBODIMENTS

The following description of the preferred embodiment(s) is merely exemplary in nature and is in no way intended to limit the invention, its application, or uses.

### Materials and Methods

### Antibodies and reagents

Mouse monoclonal antibodies raised against vinculin (Sigma), Rac1, PKCα, PKCδ, and PKCε (BD Transduction Labs) were used according to the manufacturer's instructions. Mouse monoclonal antibody raised against active human β₁ integrin (12G10) was used as described previously (Mould et al., 1998). Rat monoclonal antibody raised against inactive human β₁ integrin (mab13) was a gift from Ken Yamada (NIH, USA). Cy2-conjugated anti-mouse IgG was purchased from Jackson (Stratech Scientific), Alexa Fluor680-conjugated anti-mouse IgG and TRITC-conjugated phalloidin from Molecular Probes (Invitrogen). Recombinant fibronectin polypeptides encompassing type III repeats 6-10 (50K) and 12-15 (H/0) were expressed as recombinant polypeptides as described previously (Makarem et al., 1994), and poly-L-lysine (PLL) was purchased from Sigma. The plasmid encoding the GST-PAK-1 CRIB domain was a gift from Professor Kozo Kaibuchi (Nagoya University School of Medicine, Japan).

### Cell Culture

The generation of immortalized wild-type and syndecan-4−/− MEFs has been described previously (Bass et al., 2007b). To allow expression of the large T antigen, the MEFs were cultured at 33° C. in DME (Sigma) supplemented with 10% fetal bovine serum, 2 mM L-glutamine and 20 U/ml IFNγ (Sigma). One to two days before each experiment, cells were passaged to ensure an active proliferative state. K562 human lymphocytes were cultured in RPMI1640 (Lonza) supplemented with 10% fetal bovine serum, 2 mM L-glutamine.

### Cell Spreading and Adhesion Complex Formation Assays

For immunofluorescence, 13-mm diameter glass coverslips, placed into 6-well plates, were derivatized for 30 minutes with 1 mM sulpho-m-maleimidobenzoyl-N-hydrosuccinimide ester (Perbio). For spreading on mab13 monoclonal antibody, coverslips were pre-coated with 10 μg/ml goat anti-Rat IgG FC fragment (Jackson, Stratech Scientific). For biochemical assays, 6-well tissue culture-treated plastic plates (Corning BV) were coated directly with ligand. Coverslips or dishes were coated for 2 hours at room temperature with 10 μg/ml 50K, H/0, PLL or mab13 in Dulbecco's PBS containing calcium and magnesium (Lonza), and blocked with 10 mg/ml heat-denatured BSA for 30 minutes at room temperature (Humphries et al., 1986). Equivalent ligand-coating between glass and plastic was tested by ELISA using the anti-fibronectin mAb 333 (Bass et al., 2007a). To prevent de novo synthesis of ECM and other syndecan-4 ligands, cells were treated with 25 μg/ml cycloheximide (Sigma) for 2 hours prior to detachment (Couchman et al., 1983) and were then detached with 0.5 mg/ml trypsin. Cells were resuspended in DME, 25 μg/ml cycloheximide, plated at a density of 1×10⁵ cells per well, and allowed to spread at 37° C. for 2 hours. Pre-spread cells were treated with 200 nM BIM (Calbiochem) for 30 minutes if appropriate, and then stimulated with 10 μg/ml H/0 (40 min) or ultrasound (20 min), with or without pharmacological inhibitors, before fixing or preparing lysates. For extended timecourse experiments, ultrasound was only applied for the first 20 minutes of stimulation, in keeping with clinical ultrasound regimes, and cells were then maintained at 37° C. for the remainder of the duration, allowing the response to develop. For immunofluorescence, cells were fixed with 4% (w/v) paraformaldehyde, permeabilized with 0.5% (w/v) Triton X-100 diluted in PBS-, and blocked with 3% (w/v) BSA in PBS. Fixed cells were stained for vinculin and actin, mounted in Prolong®Antifade (Molecular Probes, Invitrogen Ltd, Paisley, UK) and photographed on an Olympus BX51 microscope using a 60×NA 1.40 PlanApo objective and Photometrics CoolSNAP ES camera. Images were compiled and analyzed using ImageJ software. The total area of adhesion complexes per cell was calculated by recording the area of fluorescence intensity above an empirically determined threshold, following rolling ball background subtraction. The same threshold was used for all conditions within a single experiment.

### Ultrasound Stimulation

6-well plates containing pre-spread MEFs were mounted onto an array of 6 ultrasound transducers (Exogen 2000; Smith and Nephew Inc., Memphis, Tenn.), the bases of the wells coupled to the transducers using water-based gel. The transducers generated 30 mWcm⁻² pulsed ultrasound with a 1.5 MHz wave frequency, pulsed at 1 kHz for a duration of 20 minutes.

### Flow Cytometry

For analysis of integrin activity by flow cytometry, K562 suspension cells were stimulated with ultrasound for 20 minutes, followed by centrifugation and re-suspension in ice cold media. Cells were stained with primary antibody (12G10), diluted to 10 μg/ml in Dulbecco's PBS containing calcium and magnesium (Lonza), 0.02% (w/v) sodium azide on ice for 1 hour. The cells were washed with PBS, 1% (v/v) fetal bovine serum, followed by 10 μg/ml secondary antibody diluted in PBS, 10% (v/v) fetal bovine serum for 30 minutes. For manganese stimulation all antibodies and wash buffers were supplemented with 1 mM MnCl₂. Cells were then fixed with 2% (w/v) paraformaldehyde, and analysed on a Dako Cyan flow cytometer, using an excitation wavelength of 488 nm. A 530/30 nm bandpass filter was used to detect the emissions.

### GTPase Activation Assays

Active Rac1 was affinity purified from lysates prepared in 20 mM HEPES (pH 7.4), 10% (v/v) glycerol, 140 mM NaCl, 1% (v/v) NP-40, 0.5% (w/v) sodium deoxycholate, 4 mM EGTA, 4 mM EDTA, 1 mM AEBSF, 50 μg/ml aprotinin, 100 μg/ml leupeptin using 300 μg GST-PAK CRIB domain immobilized on agarose beads. Active GTPase was eluted in SDS-sample buffer, resolved by SDS-PAGE and transferred to nitrocellulose. Transferred proteins were detected using the Odyssey Western blotting fluorescent detection system (LI-COR Biosciences UK Ltd., Cambridge, UK). This involved blocking the membranes with casein blocking buffer (Sigma) and then incubating with the anti-Rac1 primary antibody diluted 1:1000 in blocking buffer, 0.1% (v/v) Tween-20. Membranes were washed with PBS, 0.1% (v/v) Tween-20 and incubated with Alexa Fluor 680-conjugated anti-mouse IgG diluted 1:5000 in blocking buffer, 0.1% (v/v) Tween-20. After rinsing the membrane, proteins were detected using an infrared imaging system that allowed both an image of the membrane and an accurate count of bound protein to be recorded. For all experiments, equivalent loading between time points was confirmed by blotting the crude lysate for vinculin. The significance of changes in GTPase activity was established using paired Student T-tests of normally distributed small samples (n=5-10).

### RNAi Knockdown of PKCα

An siRNA duplex of sequence (sense) GAAGGGUUCUCGUAUGUCAUU (with ON TARGET™ modification for enhanced specificity), and an siGLO®, non-targetting control duplex were purchased from Dharmacon (Thermo Fisher Scientific). 0.8 nmol of oligo was transfected into a 90% confluent 75-cm² flask of wild-type MEFs using Lipofectamine™2000 reagent (Invitrogen). After 24 hours, the cells were passaged and then used for experiments after a further 24 hours. Expression of PKCα was tested by Western blotting.

### RESULTS

In order to differentiate between effects on signals downstream of α₅β₁ integrin or syndecan-4, mouse embryonic fibroblasts (MEFs) were treated with cycloheximide, to prevent de novo matrix synthesis, plated onto individual, recombinant ligands of α₅β₁ integrin and syndecan-4, and assayed for the ability to spread and form focal adhesions.

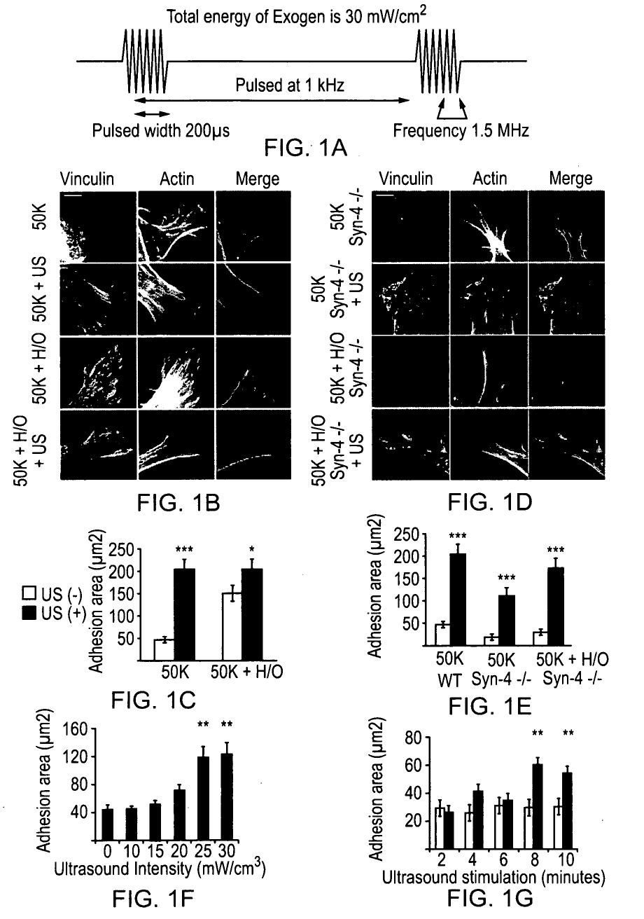

As described previously (Bass et al., 2007a), MEFs plated onto a recombinant 50 kDa fragment of fibronectin (50K), encompassing the binding sites for α₅β₁ integrin (Danen et al., 1995), failed to form vinculin-containing focal adhesions unless stimulated with a soluble syndecan-binding fragment of fibronectin, comprising type III repeats 12-15 (H/0) (FIG. 1B).

Stimulation of MEFs, prespread on 50K, with low intensity (30 mWcm⁻²), pulsed ultrasound, 1.5 MHz wave frequency, 1 kHz pulse frequency of for a duration of 20 minutes (FIG. 1A), caused the formation of vinculin-containing focal adhesions that were strikingly similar to those formed in response to syndecan-4 engagement (FIG. 1B). Focal adhesion formation was quantified by measuring the total adhesion area per cell and revealed comparable responses to H/0 and ultrasound stimuli that were not cumulative in cells subjected to both stimuli (FIG. 1C).

### Ultrasound Acts Downstream of Syndecan-4

To ascertain whether ultrasound activated syndecan-4 itself, or influenced molecules downstream of the receptor, MEFs isolated from the syndecan-4 knockout mouse were subjected to the same stimulation regime. Syndecan-4-null MEFs formed focal adhesions in response to ultrasound although, predictably, they did not respond to the soluble syndecan-4 ligand (FIG. 1D+E), indicating that ultrasound acts downstream of syndecan-4.

### Relationship Between Ultrasound Intensity and Focal Adhesion Area

The ultrasound stimulus was characterized further by varying both the intensity and duration of stimulation. The relationship between ultrasound intensity and focal adhesion area followed a sigmoid curve with an inflection point of 21.4±0.7 mWcm⁻² (FIG. 1F). The sigmoid relationship suggests that ultrasound activates a specific signaling cascade, upon reaching a particular threshold, rather than augmenting signals already occurring in the unstimulated cell. Varying the duration of stimulation gave a similar indication. In comparison with unstimulated cells, the focal adhesion area of MEFs was significantly greater following 8 minutes of stimulation (p=0.0043), but not after just 6 minutes of stimulation (p=0.341) (FIG. 1G). To ensure that the observed differences in adhesion formation were dependent on reaching a stimulus threshold, rather than the time required for development of focal adhesions, MEFs were also fixed 20 minutes after the 2-10 minute stimulation period and yielded the same result (data not shown). The concept of commitment to adhesion formation at a specific ultrasound threshold is consistent with in vivo investigations into the therapeutic benefits of ultrasound treatment, where the reduction in healing times following 30-50 mWcm⁻² ultrasound were not improved by increasing ultrasound intensity to 100 mWcm⁻² (Yang et al., 1996). From these experiments we can conclude that stimulation of a cell with ultrasound, above a 25 mWcm⁻² threshold, is sufficient to trigger a signal downstream of syndecan-4, thus dispensing with the contribution of one of the coupled prototypic fibronectin receptors during focal adhesion formation.

### Ultrasound can not Substitute for Signals Downstream of Integrin Engagement

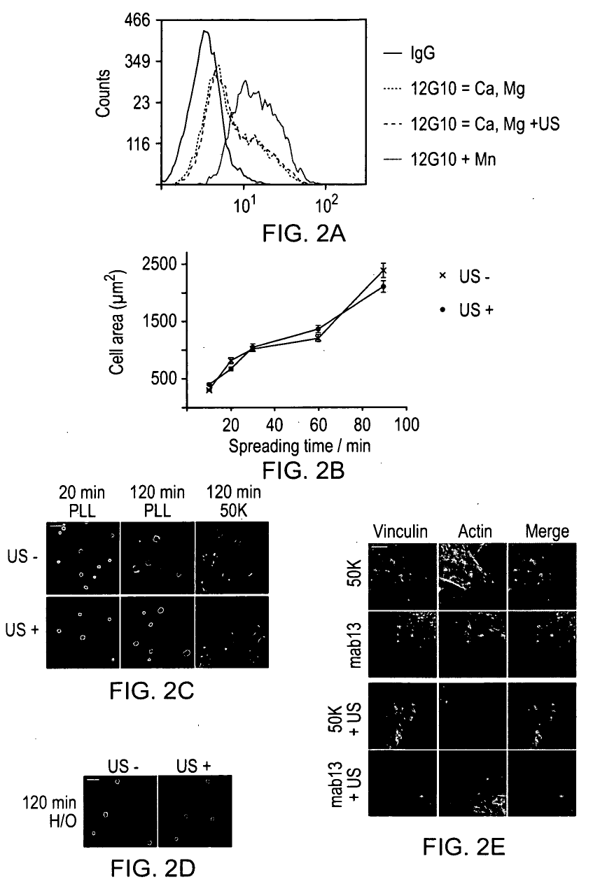

Ascertaining whether ultrasound could substitute for signals downstream of integrin engagement, during adhesion formation, was less straight forward as cells fail to spread on the syndecan-4 ligand, and a number of approaches were used. Previous investigations have shown that ultrasonic induction of molecules such as nitric oxide synthase can be blocked by inhibiting integrins (Tang et al., 2007), but it does not automatically follow that ultrasound influences integrin activity. We measured directly the activation of β₁ integrins on the cell surface by flow cytometry using a monoclonal antibody, 12G10 that specifically recognizes an activation epitope of human β₁ integrin (Mould et al., 1998). The staining of K562 lymphocytes with 12G10 could be enhanced by activating the integrin with a salt solution containing 1 mM Mn²⁺, rather than 1 mM Ca²⁺, 0.5 mM Mg²⁺ (FIG. 2A). However, ultrasound stimulation had no effect on 12G10-binding in either condition, indicating that ultrasound does not activate the integrin directly (FIG. 2A+data not shown). It has been reported previously that integrin engagement supports cell spreading through the formation of integrin clusters (Mostafavi-Pour et al., 2003) while engagement of syndecan-4 mediates the recruitment of vinculin and active Rac1 to the pre-complexes to coordinate adhesion dynamics during cell migration (Bass et al., 2007b). Ultrasound did not augment integrin-mediated events, and had no effect on the rate of integrin-mediated spreading of MEFs on 50K (FIG. 2B). Furthermore, MEFs adhering to poly-L-lysine, through weak electrostatic interactions, were smaller than those spread on fibronectin or 50K and were unaffected by ultrasound, indicating that the adhesive properties of the cell were not enhanced (FIG. 2C). In the absence of an integrin ligand, the syndecan-4 ligand, H/0, supported only weak cell attachment, and ultrasound stimulation was unable to substitute for signals downstream of the integrin to promote cell spreading (FIG. 2D). Finally, fibroblasts plated onto a monoclonal antibody that maintains β₁ integrin in an inactive conformation (mab13) spread but failed to form focal adhesions in response to ultrasound (FIG. 2E), indicating that active integrin is a requirement rather than a consequence of ultrasound action. Collectively, these experiments indicate that ultrasound acts on a signal downstream of syndecan-4, rather than α₅β₁ integrin, and equally importantly that activation of both pathways is necessary for focal adhesion formation.

Ultrasound can Trigger the Activation of Rac1 and the Formation of Focal Adhesions Through a Mechanism that is Independent of the Syndecan-4/PKCα Signaling Cascade

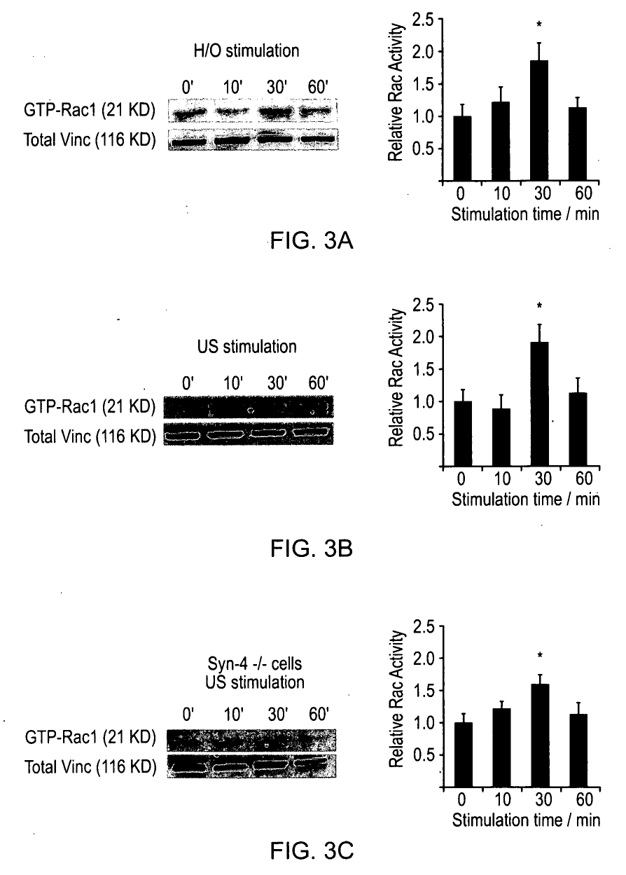

To assess the effect of ultrasound stimulation on Rac1, MEFs prespread on 50K were stimulated with either H/0 or ultrasound, and active Rac1 was affinity precipitated from lysates over a time course. Stimulating MEFs with ultrasound or the soluble syndecan-4 ligand induced similar waves of Rac1 activity that peaked at 30 minutes (FIG. 3A+B). Furthermore, stimulating syndecan-4-null MEFs with ultrasound caused a similar wave (FIG. 3C), despite the cell line already having constitutively elevated levels of GTP-Rac1, in comparison with wild type MEFs (Bass et al., 2007b; Saoncella et al., 2004).

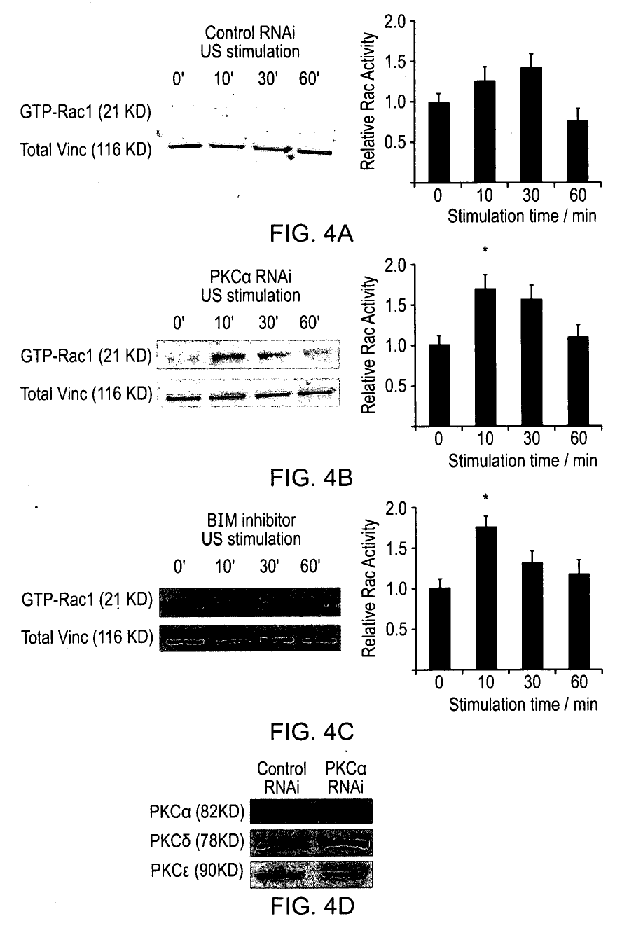

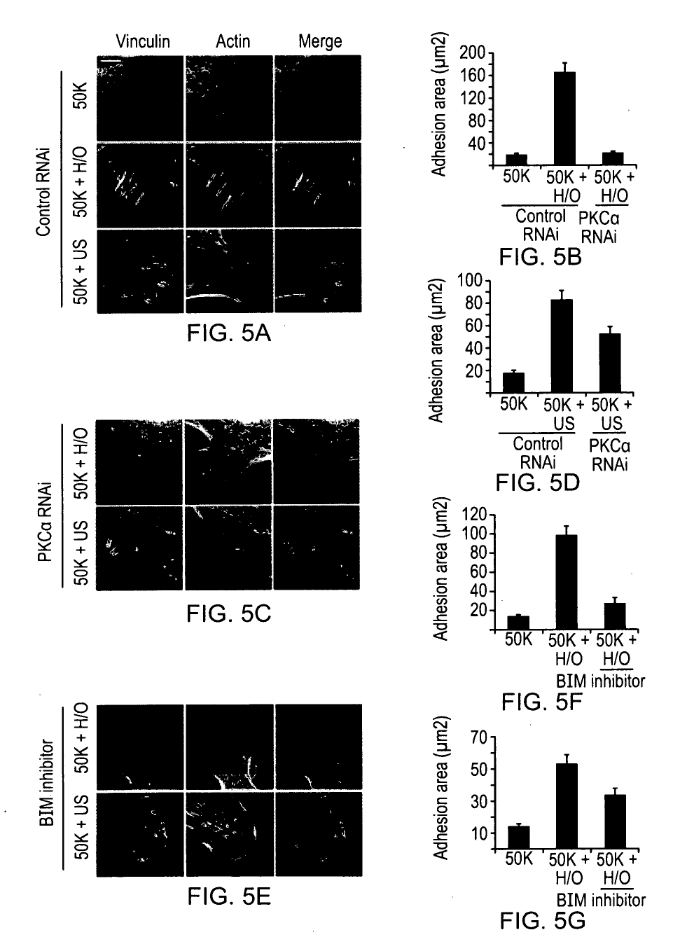

Regulation of Rac1 by syndecan-4 is known to be mediated by PKCα (Bass et al., 2007b), which is activated by direct association with the syndecan-4 cytoplasmic domain (Koo et al., 2006). The possibility that PKCα is the target of ultrasound during Rac1 regulation was tested using siRNAs and pharmacological inhibitors. Spread MEFs transfected with a non-targeting, control oligo responded to ultrasound by activating Rac1 over a similar time course to untransfected MEFs (FIG. 4A). Reduction of PKCα expression to less than 20% by RNAi (FIG. 4D), known to prevent syndecan-4-induced Rac1 activation (Bass et al., 2007b), did not block Rac1 activation in response to ultrasound (FIG. 4B). In the same way, treatment of MEFs with the PKC inhibitor, 200 nM bisindolylmaleimide I (BIM), did not prevent ultrasound-induced Rac1 activation (FIG. 4C), demonstrating that ultrasound exerts its influence downstream of the syndecan-4/PKCα signaling cascade. The PKCα-independent nature of ultrasound action was also manifested during focal adhesion formation. MEF's transfected with the non-targeting control oligo responded to either H/0 or ultrasound stimuli by forming vinculin-containing focal adhesions (FIGS. 5A,B+D). Reduction of PKCα expression by RNAi blocked the syndecan-4-mediated response to H/0 (FIG. 5B+C), but did not prevent focal adhesion formation in response to ultrasound (FIG. 5C+D). Likewise, pharmacological inhibition of PKC with BIM blocked focal adhesion formation in response to H/0, but not ultrasound (FIG. 5E+F).

The following documents are herein incorporated by reference:
Arnaout, M. A., B. Mahalingam, and J. P. Xiong. 2005. Integrin structure, allostery, and bidirectional signaling. Annu Rev Cell Dev Biol. 21:381-410.
Bass, M. D., M. R. Morgan, and M. J. Humphries. 2007a. Integrins and syndecan-4 make distinct, but critical, contributions to adhesion contact formation. Eur Phys J E Soft Matter. 3:372-376.
Bass, M. D., K. A. Roach, M. R. Morgan, Z. Mostafavi-Pour, T. Schoen, T. Muramatsu, U. Mayer, C. Ballestrem, J. P. Spatz, and M. J. Humphries. 2007b. Syndecan-4-dependent Rac1 regulation determines directional migration in response to the extracellular matrix. J Cell Biol. 177:527-538.
Bernfield, M., M. Gotte, P. W. Park, O. Reizes, M. L. Fitzgerald, J. Lincecum, and M. Zako. 1999. Functions of cell surface heparan sulfate proteoglycans. Annu Rev Biochem. 68:729-77.
Bloom, L., K. C. Ingham, and R. O. Hynes. 1999. Fibronectin regulates assembly of actin filaments and focal contacts in cultured cells via the heparin-binding site in repeat III13. Mol Biol Cell. 10:1521-36.
Cavani, A., G. Zambruno, A. Marconi, V. Manca, M. Marchetti, and A. Giannetti. 1993. Distinctive integrin expression in the newly forming epidermis during wound healing in humans. J Invest Dermatol. 101:600-604.
Chen, Y. J., C. J. Wang, K. D. Yang, P. R. Chang, H. C. Huang, Y. T. Huang, Y. C. Sun, and F. S. Wang. 2003. Pertussis toxin-sensitive Galphai protein and ERK-dependent pathways mediate ultrasound promotion of osteogenic transcription in human osteoblasts. FEBS Lett. 554:154-8.
Couchman, J. R., M. Hook, D. A. Rees, and R. Timpl. 1983. Adhesion, growth, and matrix production by fibroblasts on laminin substrates. J Cell Biol. 96:177-83.
Danen, E. H., S. Aota, A. A. van Kraats, K. M. Yamada, D. J. Ruiter, and G. N. van Muijen. 1995. Requirement for the synergy site for cell adhesion to fibronectin depends on the activation state of integrin alpha 5 beta 1. J Biol Chem. 270:21612-8.
Del Pozo, M. A., N. B. Alderson, W. B. Kiosses, H. H. Chiang, R. G. Anderson, and M. A. Schwartz. 2004. Integrins regulate Rac targeting by internalization of membrane domains. Science. 303:839-42.
Doan, N., P. Reher, S. Meghji, and M. Harris. 1999. In vitro effects of therapeutic ultrasound on cell proliferation, protein synthesis, and cytokine production by human fibroblasts, osteoblasts, and monocytes. J Oral Maxillofac Surg. 57:409-19; discussion 420.
Echtermeyer, F., M. Streit, S. Wilcox-Adelman, S. Saoncella, F. Denhez, M. Detmar, and P. Goetinck. 2001. Delayed wound repair and impaired angiogenesis in mice lacking syndecan-4. J Clin Invest. 107:R9-R14.
Elenius, K et al. 1991. Induced expression of syndecan in healing wounds. J. Cell. Biol. 114:585-595.
Gallo, R., C. Kim, R. Kokenyesi, N. S. Adzick, and M. Bernfield. 1996. Syndecans-1 and -4 are induced during wound repair of neonatal but not fetal skin. J Invest Dermatol. 107:676-83.
Gebauer, D., E. Mayr, E. Orthner, and J. P. Ryaby. 2005. Low-intensity pulsed ultrasound: effects on nonunions. Ultrasound Med Biol. 31:1391-402.
Heckman, J. D., J. P. Ryaby, J. McCabe, J. J. Frey, and R. F. Kilcoyne. 1994. Acceleration of tibial fracture-healing by non-invasive, low-intensity pulsed ultrasound. J Bone Joint Surg Am. 76:26-34.
Humphries, M. J., S. K. Akiyama, A. Komoriya, K. Olden, and K. M. Yamada. 1986. Identification of an alternatively spliced site in human plasma fibronectin that mediates cell type-specific adhesion. J Cell Biol. 103:2637-2647.
Koo, B. K., Y. S. Jung, J. Shin, I. Han, E. Mortier, P. Zimmermann, J. R. Whiteford, J. R. Couchman, E. S. Oh, and W. Lee. 2006. Structural basis of syndecan-4 phosphorylation as a molecular switch to regulate signaling. J Mol Biol. 355:651-63.
Kristiansen, T. K., J. P. Ryaby, J. McCabe, J. J. Frey, and L. R. Roe. 1997. Accelerated healing of distal radial fractures with the use of specific, low-intensity ultrasound. A multicenter, prospective, randomized, double-blind, placebo-controlled study. J Bone Joint Surg Am. 79:961-73.
Makarem, R., P. Newham, J. A. Askari, L. J. Green, J. Clements, M. Edwards, M. J. Humphries, and A. P. Mould. 1994. Competitive binding of vascular cell adhesion molecule-1 and the HepII/IIICS domain of fibronectin to the integrin alpha 4 beta 1. J Biol Chem. 269:4005-11.
Morgan, M. R., M. J. Humphries, and M. D. Bass. 2007. Synergistic control of cell adhesion by integrins and syndecans. Nat Rev Mol Cell Biol. 31:31.
Mostafavi-Pour, Z., J. A. Askari, S. J. Parkinson, P. J. Parker, T. T. Ng, and M. J. Humphries. 2003. Integrin-specific signaling pathways controlling focal adhesion formation and cell migration. J Cell Biol. 161:155-67.
Mould, A. P., A. N. Garratt, W. Puzon-McLaughlin, Y. Takada, and M. J. Humphries. 1998. Regulation of integrin function: evidence that bivalent-cation-induced conformational changes lead to the unmasking of ligand-binding sites within integrin alpha5 beta1. Biochem J. 331:821-8.
Parvizi, J., V. Parpura, J. F. Greenleaf, and M. E. Bolander. 2002. Calcium signaling is required for ultrasound-stimulated aggrecan synthesis by rat chondrocytes. J Orthop Res. 20:51-7.
Praemer, A., S. Fumer, and O. Price. 1992. Musculoskeletal conditions in the United States. Am. Acad. Orthop. Surg.:85-91.
Ridley, A. J. 2006. Rho GTPases and actin dynamics in membrane protrusions and vesicle trafficking. Trends Cell Biol. 16:522-9. Epub 2006 Sep. 1.
Saoncella, S., E. Calautti, W. Neveu, P. F. Goetinck, E. Tkachenko, E. Lutgens, R. V. Stan, and M. Simons. 2004. Syndecan-4 regulates ATF-2 transcriptional activity in a Rac1-dependent manner
Stepp, M. A et al 2002. Defects in keratinocyte activation during wound healing in the syndecan-1 deficient mouse. J. Cell. Sci. 115, 4517-4531.
Fibroblast growth factor 2 endocytosis in endothelial cells proceed via syndecan-4-dependent activation of Rac1 and a Cdc42-dependent macropinocytic pathway. J Biol Chem. 279:47172-6.
Tang, C. H., D. Y. Lu, T. W. Tan, W. M. Fu, and R. S. Yang. 2007. Ultrasound induces hypoxia-inducible factor-1 activation and inducible nitric-oxide synthase expression through the integrin/integrin-linked kinase/Akt/mammalian target of rapamycin pathway in osteoblasts. J Biol Chem. 282:25406-15. Epub 2007 Jun. 21.
Tang, C. H., R. S. Yang, T. H. Huang, D. Y. Lu, W. J. Chuang, T. F. Huang, and W. M. Fu. 2006. Ultrasound stimulates cyclooxygenase-2 expression and increases bone formation through integrin, focal adhesion kinase, phosphatidylinositol 3-kinase, and Akt pathway in osteoblasts. Mol Pharmacol. 69:2047-57. Epub 2006 Mar. 15.
Wang, F. S., Y. R. Kuo, C. J. Wang, K. D. Yang, P. R. Chang, Y. T. Huang, H. C. Huang, Y. C. Sun, Y. J. Yang, and Y. J. Chen. 2004. Nitric oxide mediates ultrasound-induced hypoxia-inducible factor-1alpha activation and vascular endothelial growth factor-A expression in human osteoblasts. Bone. 35:114-23.
Woods, A., J. R. Couchman, S. Johansson, and M. Hook. 1986. Adhesion and cytoskeletal organisation of fibroblasts in response to fibronectin fragments. Embo J. 5:665-70.
Yang, K. H., J. Parvizi, S. J. Wang, D. G. Lewallen, R.R. Kinnick, J. F. Greenleaf, and M. E. Bolander. 1996. Exposure to low-intensity ultrasound increases aggrecan gene expression in a rat femur fracture model. J Orthop Res. 14:802-9.
Zhou, S., A. Schmelz, T. Seufferlein, Y. Li, J. Zhao, and M. G. Bachem. 2004. Molecular mechanisms of low intensity pulsed ultrasound in human skin fibroblasts. J Biol Chem. 279:54463-9. Epub 2004 Oct. 12.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

What is claimed is:

1. A method of activating a member of the Rho kinase family in a subject by exposing the subject to an ultrasound signal.

2. The method according to claim 1, wherein the Rho kinase is GTPase Rac1.

3. The method according to claim 1, wherein the ultrasound signal is pulsed.

4. The method according to claim 3, wherein the ultrasound signal has a pulse frequency of 1 KHz.

5. The method according to claim 1, wherein the ultrasound signal has an intensity of at least 25 mWcm-2.

6. The method according to claim 1, wherein the ultrasound signal has a 1.5 MHz wave frequency.

7. The method according to claim 1, wherein the subject is exposed to the ultrasound for at least an 8 minute period.

8. The method according to claim 7, wherein the subject is human.

9. A method of promoting tissue repair in a subject by activating GTPase Rac1, the method comprising the step of exposing the tissue to an ultrasound signal.

10. The method according to claim 8, wherein the ultrasound signal is pulsed.

11. The method according to claim 9, wherein the ultrasound signal has a pulse frequency of 1 KHz.

12. The method according to claim 8, wherein the ultrasound signal has an intensity of at least 25 mWcm-2.

13. The method according to claim 8, wherein the ultrasound signal has a 1.5 MHz wave frequency.

14. The method according to claim 8, wherein the subject is exposed to the ultrasound for at least an eight minute period.

15. The method according to claim 8, wherein the subject is human.

16. A method of promoting the formation of focal adhesions in a subject by exposing the subject to an ultrasound signal.

17. The method according to claim 16, wherein the ultrasound signal is pulsed.

18. The method according to claim 17, wherein the ultrasound signal has a pulse frequency of 1 KHz.

19. The method according to claim 16, wherein the ultrasound signal has an intensity of at least 25 mWcm-2.

20. The method according to claim 16, wherein the ultrasound signal has a 1.5 MHz wave frequency.

21. The method according to claim 16, wherein the subject is exposed to the ultrasound for at least an eight minute period.

22. The method according to claim 16, wherein the subject is human.

23. A method of determining the likelihood of a deficient wound healing response in an individual, the method comprising the steps of:
a. determining the concentration of a syndecan in a sample of bodily fluid taken from an individual;
b. comparing the concentration with a predetermined cut-off value, said cut-off value being chosen to exclude concentrations of a syndecan associated with an optimal wound healing response in an individual, wherein a concentration below the cut-off value is indicative of the likelihood of a sub-optimal wound healing response.

24. The method according to claim 23, wherein the syndecan is syndecan-1 and/or syndecan-4.

25. A method of enhancing the wound healing response in an individual, the method comprising the steps of:
a. determining the concentration of a syndecan in a sample of bodily fluid taken from an individual;
b. comparing the concentration with a pre-determined cut-off value, said cut-off value being chosen to exclude concentrations of a syndecan associated with an optimal wound healing response in an individual, wherein a concentration below the cut-off value is indicative of the likelihood of a sub-optimal wound healing response; and
c. exposing the wound to ultrasound if the concentration of a syndecan in the bodily fluid is below the cut-off value.

26. The method according to claim 25, wherein the syndecan is syndecan-1 and/or syndecan-4.

27. A method of determining the likelihood of a deficient wound healing response in an individual, the method comprising the steps of:
a. determining the concentration of a PKCα in a sample of bodily fluid taken from an individual; and
b. comparing the concentration with a pre-determined cut-off value, said cut-off value being chosen to exclude concentrations of a PKCα associated with an optimal wound healing response in an individual, wherein a concentration below the cut-off value is indicative of the likelihood of a sub-optimal wound healing response.

28. A method of enhancing the wound healing response in an individual, the method comprising the steps of:
a. determining the concentration of a PKCα in a sample of bodily fluid taken from an individual;
b. comparing the concentration with a pre-determined cut-off value, said cut-off value being chosen to exclude concentrations of a PKCα associated with an optimal wound healing response in an individual, wherein a concentration below the cut-off value is indicative of the likelihood of a sub-optimal wound healing response; and
c. exposing the wound to ultrasound if the concentration of a PKCα in the bodily fluid is below the cut-off value.
